(19) United States
(12) Patent Application Publication
(10) Pub. No.: US 2004/0067880 A1
(43) Pub. Date: Apr. 8, 2004

(54) Prodrug therapy of liver diseases using receptor-mediated delivery of malarial circumsporozoite protein as a carrier

(76) Inventor: Macus Tien Kuo, Houston, TX (US)

Correspondence Address: Professor Macus Tien Kuo, 3520 Plumb Street, Houston, TX, 77005, US

(21) Appl. No.: 10/151,547

(22) Filed: Oct. 4, 2002

(51) Int. Cl. A61K 38/17; C07K 14/44; C07H 21/04

(52) U.S. Cl. 514/12; 536/23.5; 530/350

(57) ABSTRACT

Disclosed is a receptor-mediated protein delivery system using a ligand derived from the Region II of malarial circumsporozoite (CS) protein which recognizes receptors specifically localized on the surface of liver cells in vivo and many types of cultured cells grown in vitro. Using the present invention, a “suicidal gene product”, cytosine deaminase, has been successfully fused to CS protein. The recombinant fusion protein possesses both cell type targeting specificity of CS as well as cytosine deaminase enzymatic activity which catalyzes the conversion of prodrug 5-fluorocytosine into antitumor drug 5-fluorouracil and elicit cell killing capacity. Moreover, the fusion protein exhibits prolonged stability and sustained cell killing activity, due to the entrapment of the recombinant protein in a particular cellular (most likely endosome-lysosomal) compartment. Thus, the present invention provides technology for improved cell-type specificity and enhanced favorable pharmacokinetics of drug delivery.

### STATEMENT OF FEDERALLY SPONSORED RESEQARCH/DEVELOPMENT

The technology described in this invention was supported in part by research grants CA72040, CA79085 and CA16672 from the National Cancer Institute, U. S. A.

### CROSS REFERENCES TO RELATED APPLICATION

Lin-Lee, Y-C. et al. “Prolonged stability and sustained prodrug cell killing activity using receptor-mediated delivery of malarial circumsporozoite-cytosine deaminase fusion protein into liver cancer cells.” Mol. Cancer Therapeut. Vol. 1, (May, 2002, in press).

Ding et al., “Malarial circumsporozoite protein is a novel gene delivery vehicle to primary hepatocyte cultures and cultured cells. J. Biol. Chem. 270, No. 8:3667-3676, (February 1995).

Cerami, et al. “The basolateral domain of the hepatocyte plasma membrane bears receptor for the circumsporozoite protein of Plasmodium falciparum sporozoites.” Cell 70:1021-1033 (September 1992).

Mota M M, and Rodriguez, A. “Invasion of mammalian cells by Plasmodium sporozoites.” BioEssays 24:149-156 (February 2002).

Rich, et al. “Cell-adhesive motif in region II of malarial circumsporozoite protein. Science 249:1574-1577 (September 1990).

Huber, B E. et al. “Metabolism of 5-fluorocytosine to 5-fluorouracil in human colorectal tumor cells transduced with the cytosine deaminase gene: significant antitumor effects when only a small percentage of tumor cells express cytosine deaminase.” Proc. Natl. Acad. Sci. USA 91: 8302-8306 (August 1994).

### SEQUENCE LISTING

(1) GENERAL INFORMATION:

(A) APPLICANT: Kuo, M. T.

(B) TITLE OF INVENTION: Prodrug Therapy of Liver diseases Using Receptor-Mediated Delivery of Malarial Circumsporozoite Protein as a Carrier.

(C) NUMBER OF SEUENCES: 5

(D) CORRESPONDENCE ADDRESS;

(a) ADDRESSEE: M. Tien Kuo, Ph. D.

(b) NO. AND STREET NAME: 3520 Plumb Street

(c) CITY: Houston

(d) STATE: Texas 77005

(e) COUNTRY: United States of America

(f) ZIP: 77005

(2) COMPUTER READABLE FORM:

(a) MEDIUM TYPE: Zip disk

(b) COMPUTER: Macintosh G4

(c) OPERATING SYSTEM; OS-2

(d) SOFTWARE: Microsoft Window Word 98

(3) CURRENT APPLICATION DATA;

(a) APPLICATION NUMBER: US Unknown

### BACKGROUND OF THE INENTION

1. Field of the Invention

The present invention relates generally to the field of recombinant protein delivery system. In particular, the invention relates to receptor-mediated protein targeting to liver cancer cells. In one example, the invention relates to malarial circumsporozoite (CS) protein-mediated recombinant protein delivery of cytosine deaminase which catalyzes the biosynthesis of antitumor agent 5-fluorouracil (5-FU) from its prodrug 5-fluorocytosine ((5-FC) to elicit prodrug therapy of liver cancers including liver metastases of colorectal cancers.

2. Description of the Related Art

Hepatocellualr carcinoma (HCC) is one of the most deadly diseases. The current cure rate is less than 10 % (Sherlock and Dooley, 1993). Colorectal cancer (CRC) ranks the third in cancer incidence in the United States and is second only to lung cancer as a cause of cancer-related mortality. Of the more than 130,000 new cases diagnosed annually, 20 to 30% of the patients will die, mainly of progressive metastatic disease (Cohen et al., 1995). The most common metastatic site of colorectal cancer is liver. Liver metastases of colorectal cancer (MCC) constitute the major threat to human health. Despite intensive studies, the cure rates of HCC and MCC have not been improved for he last two decades (Sherlock and Dooley, 1993; Cohen et al., 1995). It is believed that eradicating MCC in the livers, although not a cure, should represent a major improvement in the treatment of advanced CRC.

Chemotherapy is the major treatment modality for inoperable HCC and MCC patients. While many chemotherapeutic regimens have been used in the treatments of these malignancies, however, the lack of improvement in the cure rates of these diseases indicate that the current treatment modalities are inadequate. One important factor that contribute to the chemotherapeutic failure is the lack of targeting specificity of the drug delivery systems, resulting in the server adverse side toxicity to the healthy organs. Therefore, innovative therapeutic approaches using novel delivery systems for the treatments of HCC and MCC are needed.

Receptor-mediated recombinant gene/protein delivery takes advantage of the selective uptake of these macromolecules by receptor-mediated endocytosis in almost all eukaryotic cells (Smith and Wu, 1999). Because of the cell type-specific distribution of various receptors, the delivery can be highly specific. Furthermore, in comparison with other delivery means such as viral delivery system and liposomal system, receptor-mediated recombinant protein delivery system allows greater flexibility of recombinant protein, because the protein delivery does not need to be packaged into viral or lipid capsids (Suzuki et al., 2001). This avoids tedious clonal selection and virus-production processes. These features make the system an attractive prospect for the treatments of human liver diseases in general and HCC, and MCC in particular.

The present invention of using receptor-mediated recombinant protein delivery system in prodrug therapy consists two components: a cell receptor-specific ligand and a functional protein that catalyzes the conversion of a prodrug into cytotoxic agent.

### BRIEF SUMMARY OF THE INVENTION

The present invention describes the strategy for improving chemotherapy therapy for liver cancers by providing a receptor-mediated delivery vehicle to target and deliver recombinant protein to hepatocellular carcinoma (HCC) and liver metastases of colorectal cancer (MCC) cells. In particular, the present invention concerns a recombinant fusion protein consisting of a hepatocyte/HCC/MCC targeting moiety derived from the surface protein of malarial parasite Plasmodium falciparum called circumsporozoite (CS) protein and a cytosine deaminase (CD).

Malaria is transmitted by the bite of infected female anopheles. Minutes after infection, the malarial sporozoites invade hepatocytes. This liver-specific invasion is mainly mediated by the CS protein, which densely coats the outer surface of sporozoites (Miller et al., 1994. Mota and Rodriguez, 2002). Receptor for the CS protein is predominantly distributed at the basolateral domain of hepatocytes (Nussenzweig, 1997; Cerami, et al., 1992) as well as HCC and MCC. It has been previously demonstrated that CS protein could be used as a delivery vehicle to introduce recombinant DNA into hepatocytes in cultures with the aid of adenovirus as an endosomal lysing agent (Ding et al., 1995). This invention concerns recombinant protein by fusing the bacterial cytosine deaminase (CD) to the N-terminus of CS protein. The CD enzyme catalyzes the conversion of nontoxic prodrug 5-fluorocytosine (5-FC) to the toxic metabolite 5-fluorouracil (5-FU) (Danielsen et al., 1992). The CD-CS fusion exhibits cell type-specificity similar to that of CS. More important, the CD enzyme remains stable in the cells and elicits sustained prodrug cell killing for at least several days. Because 5-FU is an antitumor agent commonly used for the treatment of hepatocellular carcinomas (HCC) and liver metastases of colorectal cancers (MCC). While the strategy could also synthesize 5-FU in the normal livers, but because 5-FU is preferentially more toxic to the proliferating HCC and MCC cells than the non-dividing normal liver cells. So the health livers are well tolerated to the 5-FU treatment. Therefore, this prodrug strategy is particularly applicable for the treatments of these liver malignant diseases.

### BRIEF DESCRIPTION OF THE DRAWINGS

FIG. 1A. SDS-PAGE analyses of recombinant CD-CS, CD, and CS proteins purified from bacterial hosts.

FIG. 1B. Schematic diagrams showing the structures of these proteins. RI and RII+ refer to different domains of the CS protein.

FIG. 2. Uptake of CD-CS in HepG2 cells. Purified [³⁵S] CD-CS protein (0.2 μM, 5×10⁴ cpm/μg) was incubated with 1×10⁵ HepG2 cells. At the different time intervals as indicated, cells were harvested and lysed with tissue solubilizer. The radioactivity was measured by a liquid scintillation counter. Closed circles and open circles represent cells harvested without acid wash and with acid wash, respectively. The open triangle is from a control in which different amounts of 1 μM E35 peptide which contains RII+amino acid sequence was added.

FIG. 3. Effects of CD-CS, CS and CD on cell viability of HepG2 cells (panel A) and HL60 cells (panel B) in the presence of 5-FC. 1×10⁵ cells were incubated with 0.64 μM each of CS, CD-CS, or CD for 4 hrs. Each culture was then divided into two sets: one set was washed extensively with regular medium and grown in regular medium containing 1 mM 5FC. The other set of cells were not washed and maintained in the same medium but adding 1 mM 5-FC. Cell viability was counted 4 days thereafter.

FIG. 4. Conversion of 5-FU in HepG2 or HL-60 cells after treatment with CD-CS and [³H] 5-FC. HepG2 or HL-60 cells were treated with 0.64 uM of CD-CS for 4 hrs. Cells were washed extensively and then cultured in regular medium containing [³H]5-FC. Two days later, cell extracts were prepared and medium was collected. [³H]5-FC and [³H]5-FU were separated by TLC chromatography. Data were expressed as percent uracil conversion from three experiments.

FIG. 5. Bystander effect of CD-CS/5-FC treatment. Different percentages of CD-CS-treated HepG2 cells (as indicated) were mixed with the untreated cells. Cells were treated with 1 mM 5-FC and % of dead cells was measured 4 days after treatment. As a control, untreated HepG2 cells were also used in the mixing experiment.

FIG. 6. Stability of CD-CS and CS proteins in HepG2 cells. HepG2 cells were treated with [³⁵S]-CD-CS protein (4.3×10⁴ cpm /μg) or CS (5×10⁴ cpm/μg) for different time intervals as indicated. Cells were harvested and cell extracts were prepared. Proteins were separated by 10% SDS-PAGE. Gels were stained by coomassie blue (panels C, D) followed by authradiography (A, B). Lane 1, molecular weight markers; lane 2, purified labeled CD-CS (panel C) or CD (panel D) proteins; lanes 3-8, cell extracts obtained at 2 h, 1, 2, 3, 5 and 7 days, respectively. (panel E) Densitometric analysis of autoradiographs shown in Panels A and B (data represent the average of two independent determinations).

FIG. 7. Persistent effect of CD-CS on cell killing of HepG2 cells. HepG2 cells were treated with 0.8 μM of CD-CS or with CD as a control for 4 hr. Cells was washed with fresh medium and cultured in the regular medium. Cultures were split once every 6 days. At different time intervals, cells were treated with 1 mM 5-FC. Cell viability was counted 4 days thereafter. Each point represents an average of three independent experiments.

FIG. 8. Distribution of radioactively labeled CD-CS into Bal/C mice at various intervals after injection through tail vain. Each determination represents four animals.

FIG. 9. Indirect immunohistochemical examinations of CS protein binding to normal livers (panels A and B) and human liver CRC metastases (panels C and D). Cryosections were incubated with CS protein with (A and C) or without (B and D) a peptide containing the 18 amino acid epitope. Binding of CS protein were detected by anti-CS protein monoclonal antibody followed by staining with FITC-labeled rabbit anti-mouse IgG. The sections were counterstained with propidium iodine.

### DETAILED DESCRIPTION OF THE INVENTION

The present invention includes a fusion protein comprising a ligand and an enzyme. As used herein, a fusion protein can be defined as one or more molecules fused by physical or chemical means. The fusion protein can be synthesized in living organisms using expression recombinant DNA technologies. The living organisms can be bacteria, yeasts, plants, animals and mammalian cells.

As used herein, the term “ligand” is intended to refer to any molecule that binds to another molecule or macromolecule. The term enzyme is intended to refer to any molecule or gene product that possesses biological function. The biological function is referred to any disease-related or health-related therapeutic or cytoprotective means in human health.

In the present invention, the ligand comprises a malarial protein known as circumspoprozoite (CS) protein. CS covers the surface membranes of malarial sporozoites. The amino acid sequence of CS protein can be divided into three domains: (from the N-terminal) region I (RI), repeat and region II+ (RII+). The function of RI is not clear. The repeat domain which consists of three copies of NANPNVDP and 21 copies of NANP, is immunodominant and confers the major antigenicity of CS protein (Nussenzweig, 1997; Miller et al., 1994). RII+ contains an evolutionarily conserved stretch of 18 amino acid residues among different species of Plasmodium, i.e., human malarial parasite Plasmodium falciparum [EWSPCSVTCNGIQVRIK], murine parasite P. berghei [EWSQCNVTCGSGIRVRKR] and P. yoelii [EWSQCSVTCGSGVRVRLR]. These amino acid sequences also share similarities with human cellular proteins, i.e. thrombospondin I [EWTRCSTRCGNGIQQRGR] and thrombospondin II [EWSSCSVTCGDGVOTROR]. (Robson, et al., 1988; Prater et al., 1991; Goundis et al., 1988). Because of these similarities, it has been suggested that the parasites can

### DETAILED DESCRIPTION OF THE INVENTION

The present invention includes a fusion protein comprising a ligand and an enzyme. As used herein, a fusion protein can be defined as one or more molecules fused by physical or chemical means. The fusion protein can be synthesized in living organisms using expression recombinant DNA technologies. The living organisms can be bacteria, yeasts, plants, animals and mammalian cells.

As used herein, the term “ligand” is intended to refer to any molecule that binds to another molecule or macromolecule. The term enzyme is intended to refer to any molecule or gene product that possesses biological function. The biological function is referred to any disease-related or health-related therapeutic or cytoprotective means in human health.

In the present invention, the ligand comprises a malarial protein known as circumspoprozoite (CS) protein. CS covers the surface membranes of malarial sporozoites. The amino acid sequence of CS protein can be divided into three domains: (from the N-terminal) region I (RI), repeat and region II+ (RII+). The function of RI is not clear. The repeat domain which consists of three copies of NANPNVDP and 21 copies of NANP, is immunodominant and confers the major antigenicity of CS protein (Nussenzweig, 1997; Miller et al., 1994). RII+ contains an evolutionarily conserved stretch of 18 amino acid residues among different species of Plasmodium, i.e., human malarial parasite Plasmodium falciparum [EWSPCSVTCNGIQVRIK], murine parasite P. berghei [EWSQCNVTCGSGIRVRKR] and P. yoelii [EWSQCSVTCGSGVRVRLR]. These amino acid sequences also share similarities with human cellular proteins, i.e. thrombospondin I [EWTRCSTRCGNGIQQRGR] and thrombospondin II [EWSSCSVTCGDGVOTROR]. (Robson, et al., 1988; Prater et al., 1991; Goundis et al., 1988). Because of these similarities, it has been suggested that the parasites can bypass the host defense mechanisms during their invasion. Moreover, because of its free of the immunodominant repeat, RII+ is nontoxic to the hosts

It is this region of the CS protein that specifically recognizes and binds to the receptors on the cell surface and confers targeting specificity (Cerami et al., 1992). The cognate receptor protein for CS is predominantly expressed in on the surface of liver, with low levels expression in intestines and kidney, but not in brain, bladder, thyroid, spleen, stomach, lung, and heart. However, a number of non-hepatocytic cell lines also express receptor recognizable by the circumsporozoite region II-containing polypeptide (Rich et al., 1990; Ding et al., 1995). These observations suggest that CS protein can be up-regulated during in vitro cell culture conditions. These observations also suggest that CS protein can be used as a ligand carrier for the delivery of recombinant proteins into these cells.

In preferred embodiments, the ligands would be proteins that recognize cell surface receptors. These include epidermal growth factor (Fan and Mendelsohn, 1998), tumor necrosis growth factors, fibroblastic growth factor, CD-28 (Schwarzenberger et al., 1996), interleukin-2 (Junbo, et al., 1999), and folate (Lee and Huang, 1996), etc. The ligands could be used to targeting malignant cells overexpressing their cognate receptors.

In preferred embodiments, the therapeutic gene products could be enzymes that convert prodrugs into drugs. These include cytosine deaminase which catalyzes the conversion of 5fluorocytosine (5-FC) into 5-fluorouracil (5-FU) (Danielsen et al., 1992), thymidine kinase which phosphorylates the prodrug ganciclovia to its cytotoxic derivatives, human β-glucuronidase which converts prodrug epirubicin-glucuronide (Houba et al., 1996; Murdter et al., 1997). Additional embodiments would be gene products of tumor suppressor genes, e.g., p53, p21, BRCA1, BRCA2, RB, NF-2, WT-1, WT-2, MEN-1, MEN-22, VHGL, MCC, and FCC, NF-1, etc. In still further embodiments, the gene products would be those involve in cell death/cell survival mechanisms, including BAX, BCL-2, BCL-XL, etc.

The fusion protein comprising cell ligand for receptor-mediated protein delivery also creates a situation that recombinant fusion protein is internalized by a cell type-specific manner. Once internalized, the fusion protein is entrapped in a particularly cellular compartment, most likely remains in the endosommal and lysosomal compartments. The exact mechanism of such entrapment remains to be investigated, but most likely due to the possibility that the recombinant fusion has changed the protein conformation of the ligand, thereby altered the normal endocytotic process. This results in enhanced protein stability because the entrapped protein is sequestered from cellular protein degradation machinery. This has been demonstrated in the prodrug strategy using CS protein fusion with cytosine deaminase (Lin-Lee, et al. 2002). Therefore, the invention is generally applicable to prolong protein stability for sustaining long-term enzymatic activities.

It is also proposed that the invention is generally applicable to any situation where one desires to introduce fusion configuration to interfere with cell growth by interfere normal receptor-mediated endocytotic physiology. Many malignant cells overexpress receptors for various growth factors (Fan and Mendelsohn, 1998). One may use fusion protein to intervene the normal receptor physiology of these overexpressed receptors, thereby controlling the growth of cancer cells.

On the other hand, the therapeutic gene may be directed to a non-cancerous disease state. For example, cystic fibrosis (CF) is caused by mutations in the gene encoding the cystic fibrosis transmembrane conductance regulator (CFTR). Furthermore, therapy of genetic diseases such as hyperchlesterolemia, phenylketonuria and hemophila could be achieved by the introduction of a corresponding gene products. The underlying deficiency of these diseases may also be alleviated by the introduction of these relevant proteins using the present invention.

Moreover, the present invention may be applicable to the treatment of infectious diseases, i.e., various forms of hepatitis. Interferon has been used in the treatments of hepatitis viral infections. However, interferon causes sever adverse side effects if administered systemically. The strategy of hepatic targeting using CS protein as a ligand could drastically reduce the toxicity of interferon otherwise delivered systematically and thus enhance the therapeutic efficacy of the treatment.

### EXAMPLE 1

### Preparation of Circumsporozoite-Cytosine Deminase (CD-CS) Fusion protein

A. Methods

1. Construction of Recombinant DNA

pCS27I C6×His encodes a CS protein with deletion of internal repeats but retaining the receptor binding domain (Region II+) and six histidine residues. This plasmid DNA also contains an isopropylthio-β-D-thiogalactoside (IPTG) regulatable promoter element followed by a ribosomal binding side in front of the CS gene and a transcriptional terminal signal from chloramphenicol acetyltransferase gene behind the translational termination codon. Cytosine deamninase gene was synthesized by polymerase chain reaction (PCR) using oligo (5′ AGTGGATCCACGTTTGTAATCGASTGGC, underscored nucleotides contain BamHI site) and oligo (5′ ACAGGATCCAATAACGCTTTACAAACA) and plasmid template of pSD112 (a gift of Dr. Jan Neuhard, University of Copenhagen, Denmark) which contains the Escherichia coli CD gene (Danielsen et al., 1992). The PCR product was purified and digested by restriction enzyme BamHI, and ligated into plasmid pCS27I C-6×His at the BamHI site which is located at the 5′ end of the CS gene. The resulting recombinant, designated pCD5-73.15.5, encoded CD-CS fusion protein with the full length CD inserted in frame after the third amino acid residue of the CS protein. Similarly, BamHI-digested PCR product was cloned into the BamHI site of a pQE-60 vector (Qiagen) and the resulting recombinant was designated as pCDP6 which also contains 6 histidine residues at the C-terminus. All the recombinant plasmids were verified by DNA sequencing. The plasmid DNA was transformed into E. coli SG13009(pREP4) hosts.

2. Expression and Purification of Recombinant CD-CS, CD, and CS Proteins

Bacterial cells carrying various recombinant plasmids were grown in one liter of 2×YT both containing 16 g bactotryptone, 10 g yeast extract, 5 gm NaCl, 100 mg ampicilin and 25 mg kanamycin for 2 to 3 hr. When the cultures reached an A600 of 0.6, 2 mM of IPTG was added to the medium. Three hours after induction, the cells were harvested by centrifugation and resuspended in 10 ml of buffer MCAC-0 (20 mM Tris-HCl, pH 8.0, 0.5 M NaCl, 10% glycerol and freshly prepared 1 mM phenylmethylsulfonyl fluoride, 1 μg/ml of aprotinin and 1 μg/ml of leupeptin). Cells were sonicated and the resulting cell lysates were centrifuged at 12, 000 rpm in the HB4 rotor of a Sorval high-speed RC5B centrifuge for 35 min. Clear lysate was mixed gently with 4 ml of Ni-NTA agarose in ice for 1 hour. After washing the agarose with buffer MCAC-20 (MCAC-0 containing 20 mM imidazole), the recombinant protein was eluted with buffer MCAC-200 (MCAC-0 plus 200 mM imidazole). Fractions containing CDCS, CD or CS were analyzed by 10% polyacrylamide gel electrophoresis, pooled, and dialyzed extensively in phosphate-buffered saline (PBS). Proteins were concentrated and frozen in aliquots at −70° C. until used. Typical yields (per liter) were 1-3 mg, and 4-5 mg of CD-CS and CD proteins, respectively.

B. Results

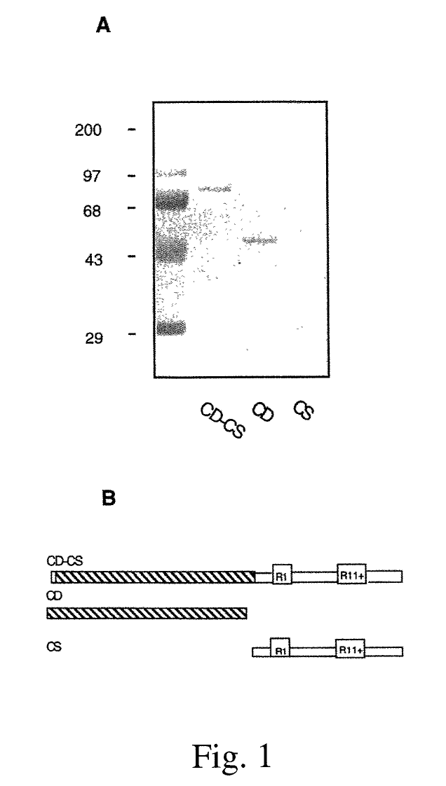

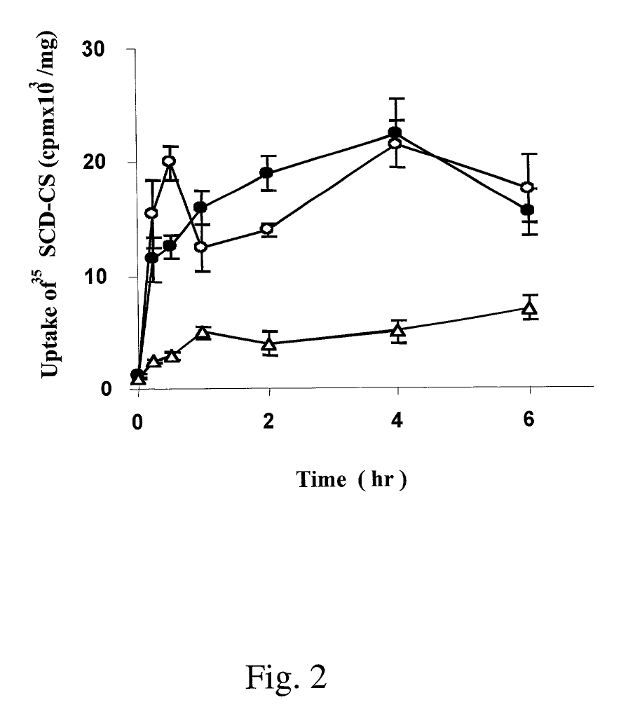

The expression of these proteins was under the control of the lac repressor. Upon induction by IPTG, these bacterial cultures produced recombinant proteins consisting 10 - 30% of total cellular proteins. These recombinant proteins contained six histidine tags at their respective C-termini. Therefore, purification of these proteins was achieved by affinity column chromatography through a Ni-NTA column. A one-step fractionation by passing the crude extracts from the induced cultures through the column resulted in 120- to 130-fold purification (data not shown). The purified proteins were then analyzed by SDS-PAGE. Single bands corresponding to the molecular mass of 45 kDa and 34 kDa were observed for the CD and CS preparations, respectively (FIG. 1A). A major band corresponding to molecular mass of 87 kDa was found in the purified CD-CS sample. This molecular mass is consistent with that for the fusion between CD and CS. CD-CS preparations also contained a minor component with molecular mass of 65 kDa. The identity of this minor band remains unknown; it may represent a degradation product of CD-CS. The specific activity of the CD-CS fusion protein, as measured by the conversion of [³H]cytosine to [³H]uracil, was 2,493 nmol/min/mg protein, about 75% that of the CD protein (3,308 nmol/min/mg). This reduction in specific activity was presumably due to the fusion. These results indicated that the recombinant fusion protein retained the enzymatic activity of CD. The repeat-deleted CS protein and CD were similarly prepared and used in parallel in the CD-CS experiments (FIG. 1B).

### EXAMPLE 2

### Dependence of Cell Type-Specificity of CD-CS-Mediated Prodrug Cell Killing

A. Methods

1. Preparation of Radioactively Labeled Recombinant Fusion Protein

[³⁵S]CD-CS-labeled protein was prepared by the method described previously (Giovane et al. 1997). In brief, bacterial cultures harboring recombinant plasmids encoding CD-CS were growth in 2×YT media until A600 of 0.6. Cells were pelleted and re-cultured in 100 ml of MEM (Life Technologies, Bethesda, Md.) supplemented with 1 mM glutamine, 25 mM HEPES, pH 7.5, 1.5 mM IPTG, and 0.1 ml of L-[³⁵S] methionine (1000 Ci/mmol. 10 mCi/ml). After culturing cells at 37° C. for an additional 2.5 hr, cells were harvested and the recombinant proteins were purified according to the procedure as described above.

2. Cytosine Deaminase Enzymatic Activity Assay

Crude cell extracts were prepared by sonicating cells in a buffer (50 mM Na phosphate, pH 7.8 and 0.3 M NaCl) followed by centrifugation. Cytosine deaminase activity was determined by measuring the production of uracil in a reaction mixture (50 μl) containing 5 mM, 6-[³H]cytosine (0.45 Ci/mol), 50 mM Tris-HCl pH 7.8, 1 mM DTT, 1 mM EDTA and cellular crude extract (10-20 μg/ml) or eluted fractions. After 1-2 hour of incubation at 37° C., 10 μl aliquots were withdrawn and analyzed by thin layer chromatography (Silica Gel 60, Selecto Scientific, Norcross, Ga.). Chromatograms were developed in 1-butanol /water (86/14, v/v). Positions of cytosine and uracil were identified by TV light. The corresponding spots for cytosine and uracil were cut out and radioactivities were determined in a scintillation counter. In some cases, 6-[3H] 5-FC (4.0 Ci/mmol) was used as a substrate in the assay.

3. Cytotoxicity Assay

Human hepatoma (HepG2), rat hepatoma (H4IIE), and mouse colorectal carcinoma MCA-26 cells were grown in Dulbecco's modified Eagle's medium (DMEM) supplemented with 10% fetal calf serum (FCS) (Life Technologies, Inc) containing penicillin (100 units/ml) and streptomycin (100 μg/ml). EL-60 cells were maintained in RPMI 1640 medium containing 10% FCS, penicillin (100 units/ml) and streptomycin (100 μg/ml). All cultures were maintained in a 37° C. incubator maintained with 5% CO₂ in air. Cells were plated in Costar 24-well culture plate at a density of 1-2×10⁵ cells/well. Cells were treated with different amounts of purified recombinant proteins for 4 hrs. The medium was then removed, cells were washed and replaced with fresh DMEM containing 1 mM 5-FC. After 3-4 days incubation, cells were gently detached by trypsin. Viable cells were determined by trypan blue exclusion staining and counted using hemacytometer.

4. Assay of [³⁵S]CD-CS Recombinant Protein Uptake

Cultured cells were incubated with 30 μl of [³⁵S]CD-CS (5×10⁴ cpm/μg). At different time intervals, cells and culture media were collected. Aliquots of collected cells and medium were resuspended in 0.1 ml of NCS II solubilizer (Amersham Corporation) and incubated at 50° C. in a water bath overnight. Radioactivities were determined by a scintillation counter. Protein contents were measured by the Bio-Rad protein assay kit.

B. Results

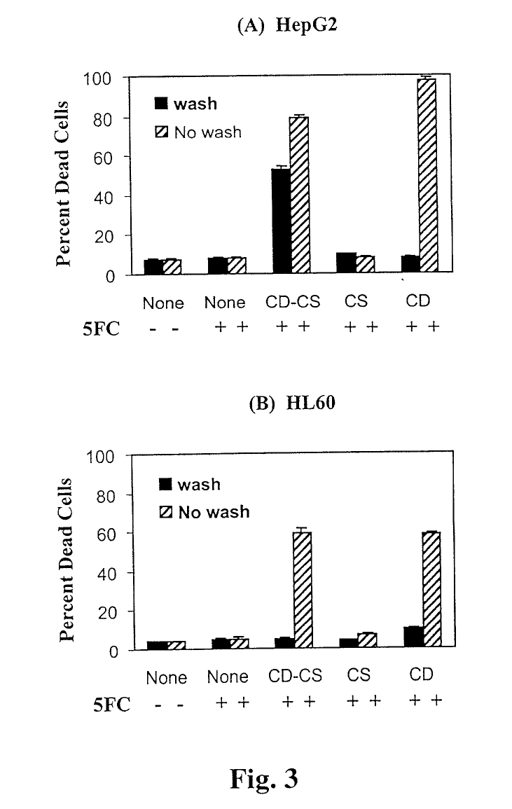

To determine whether CD-CS could be internalized by hepatoma cells, we treated HepG2 cells with [³⁵S]-labeled CD-CS protein. At different time intervals, cells were extensively washed with regular medium. Uptake of the label was determined by scintillation counting. The kinetics of protein uptake reached a plateau approximately 4 hr after the treatment. In a parallel experiment, the treated cells were further washed with a solution containing 0.25 M acetic acid. The kinetics of CD-CS uptake in the mild acid-washed cells followed the kinetics similar to that of regular washed conditions, suggesting that the CD-CS proteins were internalized, because these mild acid-washing conditions should have removed cell surface-associated ligand (Runegar et al., 1997). The uptake of recombinant protein can be competitively inhibited by a peptide (E35) containing the 23 amino acid sequence in the RII+ region that recognizes CS receptor (FIG. 2, triangles) but not the unrelated sequence (A 128, not shown). These results demonstrated that CS-CS could be internalized by hepatoma cells by a receptor-mediated mechanism. Similar results were obtained with mouse colorectal metastatic cells (MCA26).

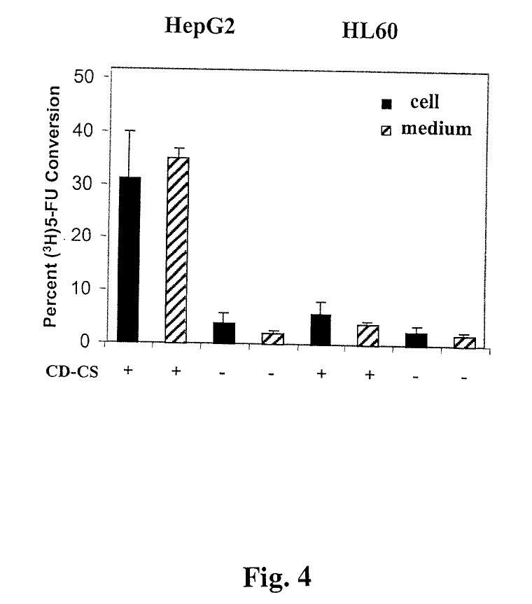

To determine whether the CD-CS protein could exert cell type-specific killing activities in the presence of exogenously added 5-FC, we performed the following experiments using a pair of cell lines, i.e., HepG2 which contains receptors for the CS protein and HL60 which does not (Ding, et al., 1995; Rich et al., 1990). First, HepG2 and HL60 cells were treated with recombinant proteins CD-CS, CS, and CD for 4 hrs. Each set of treated cells was divided into two parts: one part was extensively washed with the regular medium to remove the recombinant proteins; the other part was not. Cells in both parts were then treated with 5-FC. As expected, unwashed HepG2 cells treated with CD-CS or CD alone were sensitive to the subsequent 5-FC treatments, owing to the manufacture of cytotoxic 5-FU from 5-FC by the CD activities in the medium; the CS-treated HepG2 cells were not sensitive to 5-FC (FIG. 3A. hatched bars). In the washed cultures, only CD-CS-pretreated cells were sensitive to the treatment of 5-FC (solid bars). These results suggested that CD-CS could be taken up by HepG2 cells and exerted cell killing activity whereas CD could not. However, in similar experiments with HL-60 cells (FIG. 3B), removal of CD-CS or CD from the treated cells failed to induce cell death upon subsequent addition of 5-FC. The failure of cell killing was not due to intrinsic resistance of HL-60 cells to 5-FU, because cell death was observed in the unwashed control. These results indicate that both CD-CS and CD could not be taken up by HL-60 cells, consistent with the finding that these cells lacks CS receptor. CD-CS-mediated cell killing was concentration-dependent with an LD₅₀ for HepG2, rat hepatoma H-4-II-E, and MCA26 cells of approx. 0.22 μM (data not shown).

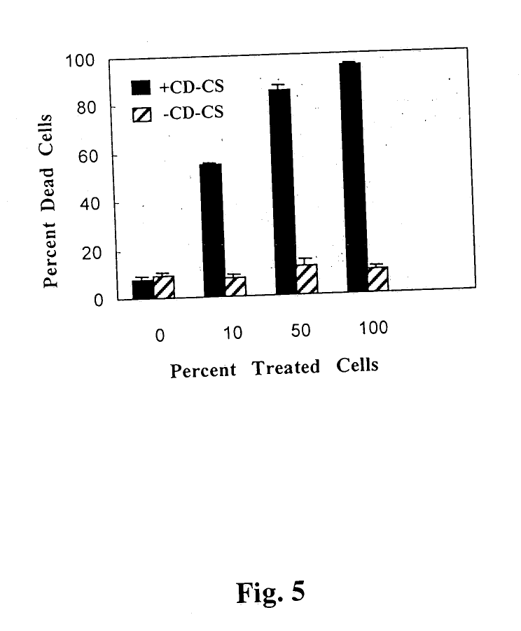

To demonstrate that the observed cell killing was related to the production of 5-FU from 5-FC, HepG2 and HL60 cells were treated with CD-CS protein for 4 hrs. Cells were then washed and incubated in regular medium supplemented with [³H]5-FC. Two days later, cell extracts were prepared. The amounts of [³H]5-FC and [³H]5-FU in the cell extracts were measured using thin-layer chromatography. Likewise, the amounts of these radioactively labels in the medium was measured. As shown in FIG. 4, >30% [³H]5-FU conversion were found in the cultured medium and in cell extracts derived from HepG2 cells, whereas only <5% conversion was seen in BL60 cells. The percentages of conversion indicated here were normalized by cell numbers. It is not likely that the substantial amounts of [³H]5-FU found in the medium was contributed significantly by cell lyses, because no apparent cell death was observed under these conditions. These results indicate that the internalized CD-CS in HepG2 cells was functional, i.e., capable of synthesis 5-FU from 5-FC. More important, the synthesized 5-FU could leak into the medium, which could exert bystander effect of cell killing (Huber et al., 1993; Huber, et al., 1994).

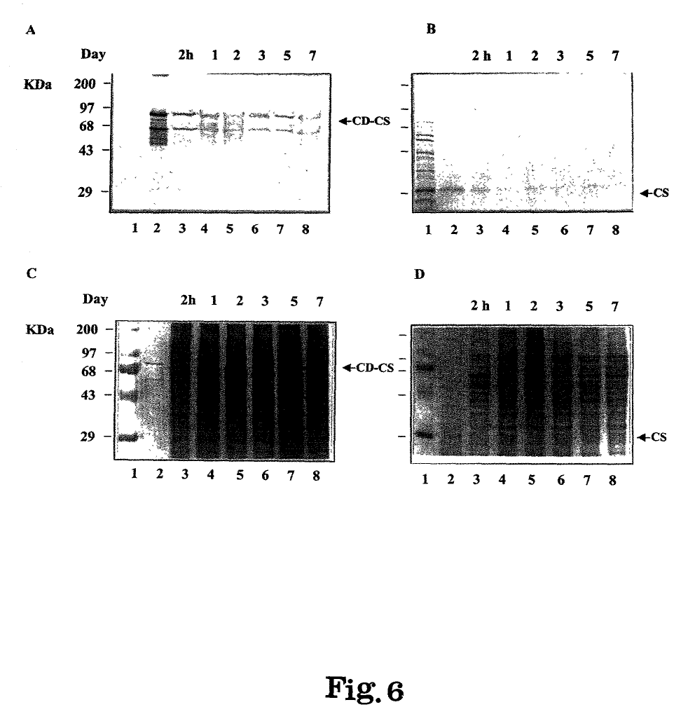

To demonstrate the bystander effect, we treated HepG2 cell with CD-CS for 4 hrs. Cells were harvested and extensively washed with the regular medium. The pretreated cells were mixed in different ratios with freshly prepared HepG2 or HL60 cells. The populations were cultured in fresh DMEM containing 5-FC for 3 days. In parallel, CD-CS-untreated cells were similarly mixed with fresh cells as controls. As shown in FIG. 5, mixed populations containing 0%, 10%, 50%, and 100% CD-CS-treated HepG2 cells resulted in 8%, 50%, 85%, and 100%, respectively, of total HepG2 cell death (FIG. 5, solid bars) whereas the controls showed less than 10% total cell death in all cases (FIG. 5, hatched bars). Similar results were obtained using HL60 cells for mixing (data not shown). Together, these results demonstrated the bystander effect of the CD-CS/5-FC strategy.

### EXAMPLE 3

### Prolonged Stability and Sustained Prodrug Cell Killing Activity Using CD-CS Fusion Protein

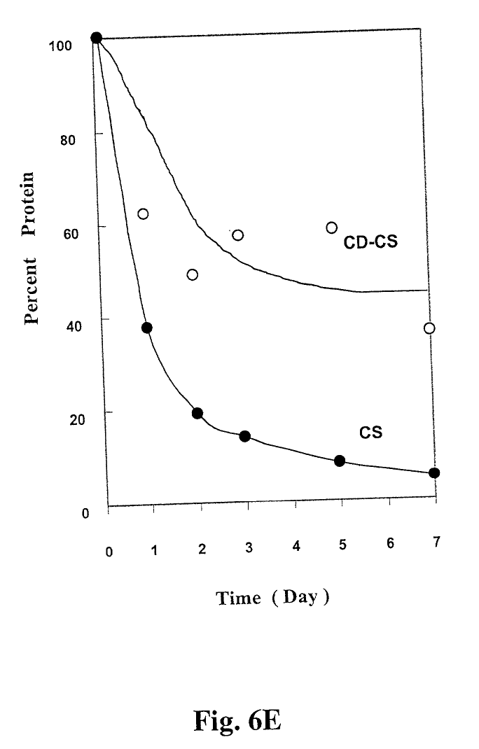

Two experiments were carried out to demonstrate the stability of the internalized CD-CS: First, confluent HepG2 cells were treated with [³⁵S]CD-CS or with [³⁵S]CS for 4 hrs. Cells were washed to remove the radioactive labels and replenished with fresh medium. At different time intervals from 2 hrs to 7 days, cell extracts were prepared and total proteins were separated by SDS-PAGE. Gels were stained with Coomassie blue to view the protein loading in different samples (FIG. 6, panels C and D) and then autoradiographed to view the stability of the labeled protein (FIG. 6, panels A and B). Densitometric analyses were used to quantify the remaining intracellularly labeled proteins using the 2-hr time point as reference of 100% (FIG. 6, panel E). These analyses revealed that the internalized CD-CS proteins were reduced approximately 50% 2 days after the delivery and remained stable thereafter for at least 7 days, whereas the internalized CS protein continued to decline to about 5% at 7 days post-delivery. These results demonstrated that the recombinant CD-CS protein exhibited enhanced intracellular stability.

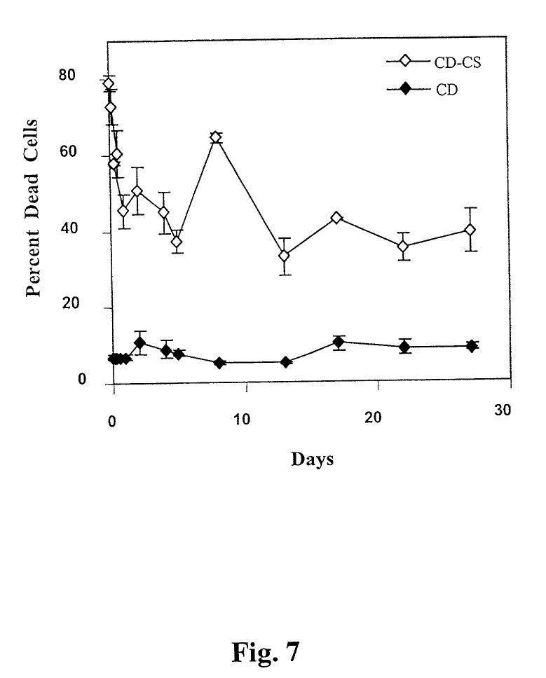

Second, to demonstrate that the internalized CD-CS remains functional several days after delivery, we carried out cell killing experiments. HepG2 cells were treated with 0.5 μM of CD-CS or CD for 4 hrs and the recombinant proteins were removed from the medium and the cells were maintained in fresh medium thereafter. At different time intervals, cells were treated with 5-FC. Sustained cell killing was observed even 28 days after the removal of the recombinant protein (FIG. 7). Since CD was not internalized, only basal level of cell killing (<10%) was observed in the CD-treated cells. These results, collectively, indicated the sustained stability and prolonged cell killing properties of the recombinant fusion protein.

### EXAMPLE 4

### Demonstration of Hepatic Targeting Specificity of CD-CS in Animals After Intravenous Injection

Previous study has demonstrated that intravenously injected recombinant CS protein containing RII+ sequence rapidly reached hepatocytes (Cerami et al., 1994). Moreover, the injected CS protein was not detected in organs including bladder, heart, kidney, lung, and spleen (Cerami et al., 1994). To demonstrate that CS can target the CD-CS fusion to the liver, we carried our investigation o the targeting profile of CD-CS fusion protein in Balb/C mice. All the recombinant proteins including radioactively labeled protein prepared from bacteria were free of endotoxin contamination. This was accomplished by passing the purified recombinant protein through an Affi-Prep Polymyxin support (Bio-Rad).

The labeled protein (20,000 cpm) was injected into the tail veins of mice. At different time intervals, animals was exsanguinated, different organs were removed, rinsed with Tris-buffered saline, blotted on filter paper, and counted for radioactivity. The amount of labeled CD-CS protein in the blood was also determined. The contribution of the labeled protein in the blood to various tissues were subtracted according to a previously described method (Cerami et al., 1994).

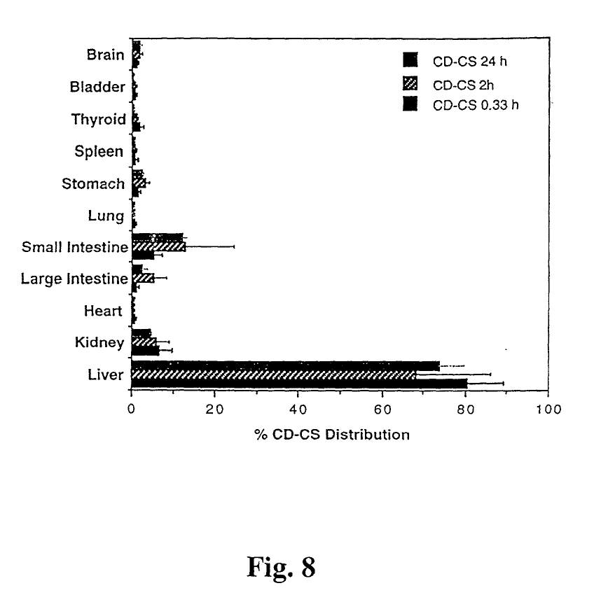

As shown in FIG. 8, it is demonstrated that 20 minutes after the injections, approximately 80% of the labeled protein were distributed in the liver. Twenty four hr later, a great majority of the counts (˜75%) remains in the livers, another 5˜10% each were in intestines and kidney, whereas the remaining were in various organs. The distribution of the labeled counts was in a general agreement with that of the labeled CS protein published previously (Cerami et al., 1994). These results indicated that CD-CS exhibits the similar targeting specificity as CS protein.

### EXAMPLE 5

### Demonstration of CS-Protein Binding to the Human Liver Metastasis of Colorectal Carcinoma Biopsy Specimens

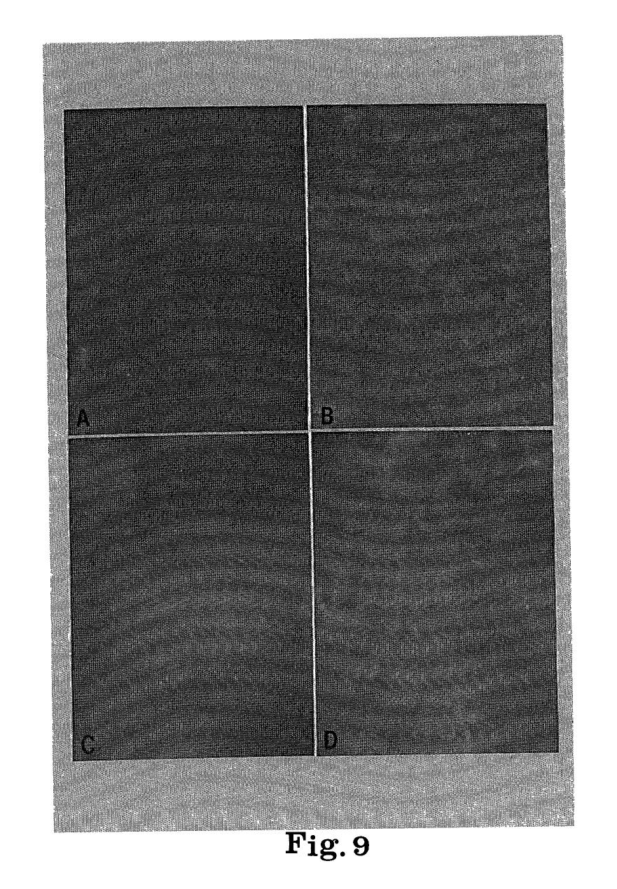

To demonstrate whether CS protein can bind to liver metastases of colorectal cancer, we prepared frozen sections of liver MCC from six patients. The tissue sections were incubated with full-length recombinant CS protein. The tissue-bound CS was probed with a monoclonal antibody (Mab2A10) reacting with the repeat domain of the ligand detected. FITC conjugated rabbit anti-mouse IgG antibody was used as secondary antibody to detect the binding. The tissue sections were counterstained by propidium iodine. By this fluorescence immunomicroscopy, strong staining was observed in the section incubated with CS protein with concentration ranging from 0.64 to 0.06 μM (FIG. 9D). The staining is mainly distributed at the surface of the cell. All 6 patients showed greater than 90% positive staining cells. Moreover, the staining could be blocked by E35 peptide containing the 18 amino acids recognized by the receptor (FIG. 9C). As a control, frozen sections of rat livers were similarly stained. In an agreement with the previous results, the staining closely followed the contours of the sinusoidal spaces of the hepatic lobules (FIG. 9B). As other controls, staining was not observed in spleen, lung, heart, where no receptors for the CS protein have been noted (not shown). These results demonstrate that MCC containing binding activities to CS protein. These results are consistent with the previous reports showing the presence of heparan sulfate proteoglycan (HSPG) in human liver metastases of colorectal cancers (Tovari et al., 1997, lozzo, et al., 1989) and CRC (Jozzo et al., 1990). Binding of CS protein to primary hepatocellular carcinomas has been noted previously (Ding et al., 1995).

As the molecular identity of CS receptor has not been fully revealed and because DNA probe or antibody for the receptor is not available at the present. It is difficult to quantify the exact amounts of CS receptor in these tissues. Nonetheless, our indirect immunoprobing shows that normal liver sections and MCC from patient biopsies display similar levels of staining intensity (FIG. 9). These results provide a justification for the development of targeted prodrug therapy of MCC.

### References

Cerami, C., Fravert, U., Sinnis, P., Takacs, B., and Nussenzweig, V. Rapid clearance of malaria circumsporozoite protein (CS) by hepatocytes. J. Exp. Med. 179:695-701, 1994.

Cerami, C., Frevert, U., Sinnis, P., Takacs, B., Clavijo, P., Santos, M. J., and Nussenzweig, V. The basolateral domain of the hepatocyte plasma membrane bears receptors for the circumsporozoite protein of Plasmodium falciparum sporozoites. Cell 70: 1021-1033, 1992.

Cohen, A. M., winawer, S. J., Friedman, M. A., and Gunderson, L. L. (ed. ) Cancer of the colon, rectum, and anaus. pp. 1-7. McGraw-Hill, Inc. New York, N.Y., 1995.

Danielsen S, Kilstrup M, Barilla K, Jochimsen B and Neuhard J, Characterization of the Escherichia coli codBA operon encoding cytosine permease and cytosine deaminase. Molec. Microbiol. 6: 1335-1344, 1992.

Ding, Z. M., Cristiano, R. J., Roth, J. A., Takacs, B., and Kuo, M. T. Malarial circumsporozoite protein is a novel gene delivery vehicle to primary hepatocyte cultures and cultured cells. J. Biol. Chem. 270: 3667-3676, 1995.

Fan, Z., and Mendelsohn, J. Therapeutic application of anti-growth factor receptor antibodies. Curr. Opin. Oncol. 10: 67-73, 1998.

Giovane, C., Schwalbach, G., and Weiss, E. In vivo labeling of overexpressed recombinant proteins in E. coli. BioTechniques 22: 796-798, 1997.

Goundis, D., and Reid, K. B. M. Properdin, the terminal complement components, thrombospondin and the circumsporozoite protein of malaria parasite contain similar sequence motifs. Nature 335:82-85, 1988.

Houba, P. H. J., Leenders, R. G. G., Boven, E., Scheeren, J. W., Pinedo, H. M., and Haisma, H. J. Characterization of novel anthracycline prodrugs activated by human γ-glucuronidase for use in antibody-direct enzyme prodrug therapy. Biochem. Pharmacol. 52:455-463, 1996.

Huber, B. E, Austin, E. A., Good, S. S., Kick, V. C., Tibbels, S., and Richards, C. A. In vivo activity of 5-fluorocytosine on human colorectal carcinoma cells genetically modified to express cytosine deaminase. Cancer Res. 53: 4619-4626, 1993.

Huber, B. E., Austin, E. A., Richards, C. A., Davis, S. T. and Good, S. S. Metabolism of 5-fluorocytosine to 5-fluorouracil in human colorectal tumor cells transduced with the cytosine deaminase gene: significant antitumor effects when only a small percentage of tumor cells express cytosine deaminase. Proc. Natl. Acad. Sci. USA 91: 8302-8306, 1994.

Iozzo, R. V. Presence of unsulfated heparan chains on the heparan sulfate proteoglycan of human colon carcinoma cells. Implications for heparan sulfate proteoglycan biosynthesis. J. Biol. Chem. 264:2690-2699, 1989.

Iozzo, R. V., Kovalszky, I., Hacobian, N., Schick, P. K., Ellingson, J. S., and Dodge, G. R. Fatty acylation of heparan sulfate proteoglycan from human colon carcinoma cells. J. Biol. Chem. 265:19980-19989, 1990.

Junbo, H., Li, Q., Zaide, W., and Yunde, H. Receptor-mediated interleukin-2 gene transfer into human hepatoma cells. Int. J. Mol. Med. 3: 600-608, 1999.

Lee, R. J., ande Huang, L. Folate-targeted, anionic liposome-entrapped polylysine-condensed DNA for tumor cell-specific gene transfer. J. Biol. Chem. 271: 8481-848, 1996.

Lin-Lee, Y. C., Nakamura, S., Gandhi, V., Curley, S. A., Stuber, D., Burkot, T. R., and Kuo, M. T. Prolonged stability and sustained prodrug cell killing activity using receptor-mediated delivery of malarial circumsporozoite-cytosine deaminase fusion protein into liver cancer cells. Mol. Cancer Therapeut. 1: xxx-xxx, 2002 (in press).

Miller, L. H., Goods, M. F., and Milon, G. Malaria pathogenesis. Science 264:1878-1883, 1994.

Mota, M. M., and Rodriguez, A. Invasion of mammalian cells by Plasmodium sporozoites. BioEsssays 24:149-156, 2002.

Murdter, T. E., Sperker, B., Kivisto, K. T., McClellan, M., Fritz, P., Fridel, G., Linder, A., Booslet, K., Toomes, H., Dierkesmann, R., and Kroemer, H. K. Enhanced uptake of doxorubicin into bronchial carcinoma: β-glucuronidase mediates release of doxorubicin from a glucoronide prodrug (HMR 1826) at the tumor site. Cancer Res. 57:2440-1445, 1997.

Nussenzweig, V. Malaria sporozoites and chylomicron remnants compete for binding sites in the liver. Behring Inst. Mitt. 99: 85-89, 1997.

Prater, C. A., Plotkin, J., Jaye, D., and Frazier, W. A. The properdin-like type I repeats of human thrombospondin contain a cell attachment site. J. Cell. Biol. 112:1031-1039, 1991.

Rich, K. A., George, F. W., IV, Law, J. L., and Martin, W. J. Cell-adhesive motif in region II of malarial circumsporozoite protein. Science 249: 1574-1577, 1990.

Robson, K. J. H., Hall, J. R. S., Jennings, M. W., Harris, T. J. R., Marsh, K., Newbold, C. I., Tate, V. E., and Weatherall, D. J. A highly conserved amino acid sequence in thrombospondin, properdin and in proteins from sporozoites and blood stages of a human malaria parasite. Nature 335:79-82, 1988.

Runegar, M., Wei, X., Berndt, N., and Hamm-Alvarex, S. Transferrin receptor recycling in rat hepatocytes is regulated by protein phosphatase 2A, possibly through effects on microtubule-dependent transport. Hepatology 26: 176-185, 1997.

Schwarzenberger, P., Spence, S. E., Gooya, J. M., Michiel, D., Curiel, D. T., Ruscetti, F. W., and Keller, J. R. Gene transfer of multidrug resistance into a factor-dependent human hematopoietic progenitor cell line: in vivo model for genetically transferred chemoprotection. Blood 87: 472-478, 1996.

Sherlock, S., and Dooley, J. Hepatic tumors, in “Disease of the liver and Bilary System.” Backwell Scient. Pub. Lond. pp. 508-531, 1993.

Smith, R. M. and Wu, G. Y. Hepatocyte-directed gene delivery by receptor-mediated endocytosis. Semin. Liver Dis. 19: 83-92, 1999.

Suzuki, M., Matsuse, T., Isigatsubo, Y. Gene therapy for lung diseases: development in the vector biology and novel concepts for gene therapy applications. Curr. Mol. Med. 1:67-79, 2001.

Tovari, J., Paku, S., Raso, E., Pogany, G., Kovalszky, I., Ladanyi, A., Lapis, K., and Timar, J. Role of sinusoidal heparan sulfate proteoglycan in liver metastasis formation. Int. J. Cancer 71:825-831, 1997.

1. A fusion protein comprising:
(i) a ligand comprising a circumsporozoite region II-containing polypeptide; and
(ii) a functional protein.

2. A ligand of claim 1, wherein said polypeptide is selected from the group consisting with amino acid sequence EWSPCSVTCGNGIQVRIK (SEQ ID NO:1, from Plasmodium falciparum parasite), and related peptides EWSQCSVTCGSGVRVRKR (SEQ ID: NO;2, from Plasmodium berghei); EWSQCSVTCGSGVRVRKR (SEQ. ID. NO:3 from Plasmodium yoelii), and EWTRCSTRCGSGVRVRKR (SEQ. ID. NO. 4 from human thrombospondin I) and PWSSCSVTCGDGVITRIR (SEQ. ID. NO. 5 from human thrombospondin II). These peptides interact with receptors on the cell surface of hepatic cells.

3. The fusion protein of claim 1, wherein said ligand is a group of growth factors that interact with their cognate receptors abnormally expression in malignant cells.

4. The fusion protein of claim 3, wherein said growth factors include epidermal growth factor, tumor necrotic factor, folate, fibroblast growth factor, etc

5. The fusion protein of claim 1, wherein said functional protein is any gene product that exhibits therapeutic values for various liver diseases.

6. The gene product of claim 5, wherein said gene product is cytosine deaminase.

7. The gene product of claim 5 wherein said gene product is inteferone.

8. The gene product of claim 5, wherein said gene product is proapoptitic proteins, including Bax, Bid, etc. or anti-apoptotic protein, including Bcl-xl, Bcl- etc.

9. The gene product of claim 5, wherein said gene product is any recombinant proteins that exhibit function to correct gene defects in the livers, including phosphoenolpyruvate carboxykinase for pepck deficiency, phenylalanine hydroxylase from phenylketonuria, ornithine transcarbamylase for ornithine transcarbamylase deficiency and LDL for familial hyperholesterolemia, etc.

10. A pharmaceutical composition comprising
(i) a ligand comprising a circumsporozoie region II-containing polypeptide,
(ii) a protein comprising said therapeutic gene product

11. A kit comprising:
(i) a ligand comprising a circumsporozoite region II-containing polypeptide; and
(ii) a recombinant protein.

12. A method of using receptor-mediated fusion protein to enhance protein stability thereby increased pharmacokinetics of drug stability and drug availability.


SEQUENCE LISTING (5 sequences)

SEQ ID NO: 1 - protein, 18 aa, Plasmodium falciparum
  SeqID1 (1)..(18)
  ref <301>: AUTHORS: Bowman S., et al.
  ref <302>: TITLE: The complete nucleotide sequence of chromosome 3 of Plasmodium falciparum.
  ref <303>: JOURNAL: Nature
  ref <304>: VOLUME: 400
  ref <305>: ISSUE: 6744
  ref <306>: PAGES: 532-538
  ref <307>: DATE: 1999-08-05
  ref <308>: DATABASE ACCESSION NUMBER: CAB38998
  ref <309>: DATABASE ENTRY DATE: 2003-01-29
  ref <313>: RELEVANT RESIDUES: (330)..(347)
EWSPCSVTCGNGIQVRIK

SEQ ID NO: 2 - protein, 18 aa, Plasmodium berghei
  SeqID2 (1)..(18)
  ref <301>: AUTHORS: Eichinger DJ, et al.
  ref <302>: TITLE: Circumsporozoite protein of Plasmodium berghei: gene cloning and identification of the immunodominant epitopes.
  ref <303>: JOURNAL: Mol. Cell. Biol.
  ref <304>: VOLUME: 6
  ref <305>: ISSUE: 11
  ref <306>: PAGES: 3965-3972
  ref <307>: DATE: 1986-11-02
  ref <308>: DATABASE ACCESSION NUMBER: M14135
  ref <309>: DATABASE ENTRY DATE: 1994-03-14
  ref <313>: RELEVANT RESIDUES: (274)..(291)
EWSQCSVTCGSGVRVRKR

SEQ ID NO: 3 - protein, 18 aa, Plasmodium yoelii
  SeqID3 (1)..(18)
  ref <301>: AUTHORS: Lal, A.A., et al.
  ref <302>: TITLE: Structure of the gene encoding the circumsporozoite protein of Plasmodium yoelii. A rodent model for examining antimalarial sporozoite vaccines.
  ref <303>: JOURNAL: J. Biol. Chem.
  ref <304>: VOLUME: 262
  ref <305>: ISSUE: 7
  ref <306>: PAGES: 2937-2940
  ref <307>: DATE: 1987-03-05
  ref <308>: DATABASE ACCESSION NUMBER: J02695
  ref <309>: DATABASE ENTRY DATE: 1994-03-14
  ref <313>: RELEVANT RESIDUES: (302)..(319)
EWSQCSVTCGSGVRVRKR

SEQ ID NO: 4 - protein, 18 aa, Homo Sapiens
  SeqID4 (1)..(18)
  ref <301>: AUTHORS: Hennessy S.W., et al.
  ref <302>: TITLE: Complete thrombospondin mRNA sequence includes potential regulatory sites in the 3′ untranslated region.
  ref <303>: JOURNAL: J Cell Biol.
  ref <304>: VOLUME: 108
  ref <305>: ISSUE: 2
  ref <306>: PAGES: 729-736
  ref <307>: DATE: 1989-02-02
  ref <308>: DATABASE ACCESSION NUMBER: NM_003246
  ref <309>: DATABASE ENTRY DATE: 2003-04-07
  ref <313>: RELEVANT RESIDUES: (443)..(460)
PWSSCSVTCGDGVITRIR

SEQ ID NO: 5 - protein, 18 aa, Homo Sapiens
  SeqID5 (1)..(18)
  ref <301>: AUTHORS: LaBell, T.L., et al.
  ref <302>: TITLE: Sequence and characterization of the complete human thrombospondin 2 cDNA: potential regulatory role for the 3′ unmtranslated region.
  ref <303>: JOURNAL: Genomics
  ref <304>: VOLUME: 17
  ref <305>: ISSUE: 1
  ref <306>: PAGES: 225-229
  ref <307>: DATE: 1993-07-02
  ref <308>: DATABASE ACCESSION NUMBER: L12350
  ref <309>: DATABASE ENTRY DATE: 1993-12-30
  ref <313>: RELEVANT RESIDUES: (445)..(462)
PWSSCSVTCGVGNITRIR